(19) United States
(12) Patent Application Publication
(10) Pub. No.: US 2026/0183576 A1
(43) Pub. Date: Jul. 2, 2026

(54) ULTRASOUND THERAPY WITH HARMONIZED ENERGY DELIVERY

(71) Applicant: Vibrato Medical, Inc., Newport Coast, CA (US)

(72) Inventors: Derek Daw, Huntington Beach, CA (US); Babak Nazer, Newport Coast, CA (US)

(21) Appl. No.: 19/007,255

(22) Filed: Dec. 31, 2024

(51) Int. Cl. A61N 7/02; G16H 40/63

(52) U.S. Cl. CPC A61N 7/02; G16H 40/63

(57) ABSTRACT

A method includes providing a patient interface comprising a transducer array and a memory, determining, for each transducer of the transducer array, a transducer power output profile. The transducer power output profile describes a relationship between a drive voltage applied to the transducer and an output power level produced by the transducer in response to the drive voltage. The method also includes storing the transducer power output profile of each transducer of the transducer array in the memory of the patient interface.

### BACKGROUND

Peripheral arterial disease (PAD) is a highly prevalent condition, affecting over 200 million people worldwide and 15-20 million people in the USA, of whom approximately 11% suffer from chronic limb-threatening ischemia (CLTI), the more advanced form of PAD with high incidence of amputation, infection, gangrene and hospitalization.

Current treatments for PAD have demonstrated limited efficacy. Patients with asymptomatic PAD can be treated with anti-platelet medications (e.g., aspirin, clopidogrel) and cholesterol-lowering statin medications in an attempt to reduce lower extremity atherosclerotic burden and to treat concomitant coronary artery disease (CAD). However, approximately 75% progress to develop symptoms of claudication or CLTI. Patients with claudication have been shown to benefit from supervised exercised therapy and the medication cilostazol, however approximately 30% progress with worsening claudication, or to develop CLTI. Revascularization with bypass surgery, angioplasty or stent implantation is recommended for refractory claudication or CLTI but is associated with high rates of restenosis and the need for repeated interventions.

Acoustic energy modalities such as shock wave therapy (SWT) and therapeutic ultrasound (TUS; also referred to in prior literature as high-intensity focused ultrasound (HIFU) or low-intensity pulsed ultrasound (LIPUS)) have been shown to promote angiogenesis, vasodilation and improve perfusion in CAD. SWT and TUS could possibly have similar effects in PAD. However, the above therapies require multiple treatments with devices that require positioning and application by a healthcare provider. This potentially limiting clinical use due to time and human resources requirement may merely provide temporary increases in blood flow via vasodilation. More efficacious systems and methods are needed, including systems that can be comfortably used at home by patients for extended durations and provide relatively long-term clinical benefits such as increased blood flow and symptomatic relief.

### SUMMARY

Systems that utilize TUS technology may include a plurality of embedded transducers configured to deliver energy to a target site. For example, an ultrasound-based sleeve may include an array of transducers embedded within the fabric of the sleeve, configured to target a patient's calf, thigh, or arm. However, due to manufacturing variability, transducers of a TUS array may not always provide the same power output, even when driven at the same drive voltage. This may lead to uneven delivery of energy by a TUS array to a target site. Therefore, characterization of individual transducers within an array may provide harmonized energy delivery from highly variable transducers.

In addition, variability of patients' anatomy and positioning of individual transducer may result in variability in the target site. For example, an ultrasound transducer positioned in a mid-section of a calf might result in a different distance to the target tibial artery than a transducer positioned at the bottom of the calf. Variability in calves' sizes and circumferences from patient to patient also account for variations in distances from a tissue target to a transducer placed at patient skin.

Limitations on efficiency is another issue resulting from inherent manufacturing limitations. For example, transducers of an array may be characterized by varying efficiencies (e.g., more efficient transducers provide more power at a given voltage, while less efficient transducers provide less power at the given voltage). Because efficiency values may be different for each transducer of the transducer array, a random charge sequence may require increasing drive voltages in some drive sequences and decreasing drive voltages in other drive sequences. However, because energy is typically stored in a capacitor or other storage medium, additional complexity is required to increase a drive voltage and subsequently decrease the drive voltage. For example, it may be required to “bleed” or dissipate some of the energy between transducer activation, which can cause delays in providing therapy and an inefficient use of drive voltage. Accordingly, it would be advantageous to activate the transducers from most efficient to least efficient to minimize delay and voltage loss between sequential transducer activations.

Aspects of the present disclosure relate to providing harmonized energy delivery from highly variable transducers in an array of a ultrasound-based sleeve placed extracorporeally and activating the transducers in an optimal sequence based on efficiency.

In some aspects, the techniques described herein relate to a method, including: providing a patient interface including a transducer array and a memory; determining, for each transducer of the transducer array, a transducer power output profile, wherein the transducer power output profile describes a relationship between a drive voltage applied to the transducer and an output power level produced by the transducer in response to the drive voltage; and storing the transducer power output profile of each transducer of the transducer array in the memory of the patient interface.

In some aspects, the techniques described herein relate to a method, wherein the transducer power output profile includes a table, a database, or a formula.

In some aspects, the techniques described herein relate to a method, wherein the transducer power output profile describes a relationship between the drive voltage applied to the transducer at a frequency and an output power level produced by the transducer in response to the drive voltage.

In some aspects, the techniques described herein relate to a method, wherein the transducer power output profile describes a relationship between a range of drive voltages applied to the transducer and output power levels produced by the transducer in response to the range of drive voltages.

In some aspects, the techniques described herein relate to a method, wherein the transducer power output profile describes a relationship between the drive voltage applied to the transducer at a frequency and an output power level produced by the transducer in response to the drive voltage.

In some aspects, the techniques described herein relate to a method, further including determining, for the transducer of the transducer array, a transducer depth profile, wherein the transducer depth profile describes a relationship between a drive frequency applied to the transducer and a depth of penetration achieved by the transducer in response to the drive frequency.

In some aspects, the techniques described herein relate to a method, further including determining an applied drive voltage for the transducer based upon the transducer power output profile and a user input.

In some aspects, the techniques described herein relate to a method, wherein the user input includes a desired power level.

In some aspects, the techniques described herein relate to a method, further including driving the transducer at the desired power level.

In some aspects, the techniques described herein relate to a patient interface, including: a connector, configured to connect the patient interface to a therapeutic ultrasound system; a transducer array, including a plurality of transducers, wherein the transducer array is in electrical communication with the connector; and a memory, configured to store information associated with each transducer of the transducer array, and wherein the memory is in electrical communication with the connector; and wherein the information associated with each transducer of the transducer array includes a plurality of transducer power output profiles, each transducer power output profile describing a relationship between a drive voltage to be applied to the transducer and an output power level produced by the transducer in response to the drive voltage.

In some aspects, the techniques described herein relate to a patient interface, wherein each transducer power output profile includes a table, a database, or a formula.

In some aspects, the techniques described herein relate to a patient interface, wherein each transducer power output profile describes a relationship between the drive voltage applied to each transducer at a frequency and an output power level produced by the transducer in response to the drive voltage.

In some aspects, the techniques described herein relate to a patient interface, wherein each transducer power output profile describes a relationship between a range of drive voltages applied to each transducer and output power levels produced by the transducer in response to the range of drive voltages.

In some aspects, the techniques described herein relate to a method, including: receiving a user input corresponding to a desired output power level; receiving from a memory, a transducer power output profile associated with a transducer of a transducer array, wherein the transducer power output profile describes a relationship between a drive voltage applied to the transducer and an output power level produced by the transducer in response to the drive voltage; determining an applied drive voltage for the transducer based upon the transducer power output profile and the desired output power level; and driving the transducer of the transducer array at the applied drive voltage.

In some aspects, the techniques described herein relate to a method, wherein the transducer power output profile includes a table, a database, or a formula.

In some aspects, the techniques described herein relate to a method, wherein the transducer power output profile describes a relationship between the drive voltage applied to the transducer at a frequency and an output power level produced by the transducer in response to the drive voltage.

In some aspects, the techniques described herein relate to a method, further including determining a plurality of applied drive voltages for the transducer based upon the transducer power output profile and a user input.

In some aspects, the techniques described herein relate to a method, further including determining an efficiency value associated with each transducer of the transducer array.

In some aspects, the techniques described herein relate to a method, wherein determining the applied drive voltage for the transducer based upon the transducer power output profile and the desired output power level includes determining the applied drive voltage for the transducer based upon the efficiency value associated with the transducer and the desired output power level.

In some aspects, the techniques described herein relate to a method, wherein driving the transducer of the transducer array at the applied drive voltage includes initially driving the transducer of the transducer array at a reduced applied voltage that is less than the applied voltage.

In some aspects, the techniques described herein relate to a method, further including increasing the reduced applied voltage over a ramp-up time period and driving the transducer of the transducer array at the increased, reduced applied voltage during the ramp-up time period.

In some aspects, the techniques described herein relate to a method, wherein the ramp-up time period is 3 min, 5 min, 7 min, 10 min, 15 min or 30 min.

In some aspects, the techniques described herein relate to a method, wherein increasing the reduced applied voltage includes increasing the reduced applied voltage by 5%, 10%, 15%, 20%, 25%, 30%, 35%, 40%, 45%, or 50% of the applied drive voltage.

In some aspects, the techniques described herein relate to a method, wherein increasing the reduced applied voltage includes increasing the reduced applied voltage by 5%, 10%, 15%, 20%, 25%, 30%, 35%, 40%, 45%, or 50% of the reduced applied drive voltage.

In some aspects, the techniques described herein relate to a method, wherein increasing the reduced applied voltage includes increasing the reduced applied in constant amounts in steps during the ramp-up time period.

In some aspects, the techniques described herein relate to a method, wherein increasing the reduced applied voltage includes increasing the reduced applied in non-constant amounts in steps during the ramp-up time period.

In some aspects, the techniques described herein relate to a method, including: providing a patient interface including a transducer array and a memory; determining, for each transducer of the transducer array, an efficiency value, wherein the efficiency value corresponds to a relationship between an output power of each transducer at a particular input drive voltage; and storing the efficiency value of each transducer of the transducer array in the memory of the patient interface.

In some aspects, the techniques described herein relate to a method, further including providing the efficiency value for each transducer of the transducer array to a therapeutic ultrasound system.

In some aspects, the techniques described herein relate to a method, wherein determining the efficiency value includes determining a ratio of the output power to the input drive voltage.

In some aspects, the techniques described herein relate to a patient interface, including: a connector, configured to connect the patient interface to a therapeutic ultrasound system; a transducer array, including a plurality of transducers, wherein the transducer array is in electrical communication with the connector; and a memory, configured to store information associated with each transducer of the transducer array, and wherein the memory is in electrical communication with the connector; and wherein the information associated with each transducer of the transducer array includes a plurality of efficiency values, each efficiency value corresponding to a relationship between an output power of each transducer at a particular input drive voltage.

In some aspects, the techniques described herein relate to a patient interface, wherein the efficiency value includes a ratio of the output power to the input drive voltage.

In some aspects, the techniques described herein relate to a method, including: receiving from a memory, an efficiency value associated with each transducer of a transducer array, wherein the efficiency value corresponds to a relationship between an output power of each transducer at a particular input drive voltage; determining a transducer activation sequence based upon the efficiency values of each transducer of the transducer array, and sequentially activating each transducer of the transducer array according to the transducer activation sequence.

In some aspects, the techniques described herein relate to a method, wherein the transducer activation sequence is based upon the efficiency value of each transducer of the transducer array from a highest efficiency value to a lowest efficiency value

In some aspects, the techniques described herein relate to a method, wherein activating includes providing a transducer with a control signal having a specified frequency for a specified duration.

In some aspects, the techniques described herein relate to a method, wherein activating each transducer occurs during a ramp-up period.

In some aspects, the techniques described herein relate to a method, wherein determining the transducer activation sequence includes determining the transducer activation sequence based upon the efficiency value of each transducer of the transducer array from a highest efficiency value to a lowest efficiency value.

In some aspects, the techniques described herein relate to a method, wherein determining the transducer activation sequence includes determining the transducer activation sequence based upon the efficiency value of each transducer of the transducer array from a lowest efficiency value to a highest efficiency value.

In some aspects, the techniques described herein relate to a method, further including storing the transducer activation sequence in a memory.

In some aspects, the techniques described herein relate to a method, wherein sequentially activating each transducer includes sequentially driving each transducer of the transducer array at an initial applied drive voltage that is less than a final applied voltage.

In some aspects, the techniques described herein relate to a method, further including determining an increased applied voltage over a ramp-up time period, wherein the increased applied voltage is greater than the initial drive voltage and driving the transducer of the transducer array at the increased applied voltage during the ramp-up time period.

In some aspects, the techniques described herein relate to a method, wherein the ramp-up time period is 3 min, 5 min, 7 min, 10 min, 15 min or 30 min.

In some aspects, the techniques described herein relate to a method, wherein determining an increased applied voltage includes increasing the initial applied voltage by 5%, 10%, 15%, 20%, 25%, 30%, 35%, 40%, 45%, or 50% of the initial applied voltage.

In some aspects, the techniques described herein relate to a method, wherein increasing the initial applied voltage includes increasing the initial applied voltage by 5%, 10%, 15%, 20%, 25%, 30%, 35%, 40%, 45%, or 50% of the final applied voltage.

In some aspects, the techniques described herein relate to a method, wherein increasing the initial applied voltage includes increasing the initial applied voltage in constant amounts in steps during the ramp-up time period.

In some aspects, the techniques described herein relate to a method, wherein increasing the initial applied voltage includes increasing the initial applied voltage in non-constant amounts in steps during the ramp-up time period.

In some aspects, the techniques described herein relate to a method, including: determining a target tissue treatment depth or target tissue thickness associated with a transducer of a transducer array; providing a first drive signal to the transducer, the first drive signal having a first frequency; and providing a second drive signal to the transducer, the second drive signal having a second frequency different than the first frequency, when the target tissue thickness exceeds a predetermined threshold.

In some aspects, the techniques described herein relate to a method, wherein determining the target tissue treatment depth or target tissue thickness includes receiving a user input.

In some aspects, the techniques described herein relate to a method, wherein the user input includes one or more of data, an image, a formula or a table.

In some aspects, the techniques described herein relate to a method, wherein determining the target tissue treatment depth or target tissue thickness includes receiving a patient parameter and determining the target tissue treatment depth or target tissue thickness from the patient parameter.

In some aspects, the techniques described herein relate to a method, wherein the patient parameter includes a height, a weight, a body mass index value or a transducer position with respect to a treatment area of a patient.

In some aspects, the techniques described herein relate to a method, wherein determining the target area and tissue thickness includes receiving a measurement.

In some aspects, the techniques described herein relate to a method, wherein determining the target area and tissue thickness includes receiving information associated with the transducer of the transducer array from a memory of patient interface supporting the transducer array.

In some aspects, the techniques described herein relate to a method, wherein determining the target tissue thickness includes determining a position of the transducer within the transducer array.

In some aspects, the techniques described herein relate to a method, wherein determining the target tissue thickness includes determining a position of the transducer on a patient interface supporting the transducer array.

In some aspects, the techniques described herein relate to a method, wherein the first frequency is about 450 kHz, or is in a range of about 350 kHz to 500 kHz, or about 425 kHz to about 575 kHz.

In some aspects, the techniques described herein relate to a method, wherein the second frequency is about 500 kHz, or is in a range of about 350 kHz to 500 kHz, or about 425 kHz to about 575 kHz.

In some aspects, the techniques described herein relate to a method, wherein providing the first drive signal to the transducer includes providing the first drive signal to the transducer for a first duration.

In some aspects, the techniques described herein relate to a method, wherein providing the second drive signal to the transducer includes providing the second drive signal to the transducer for a second duration, wherein the second duration is different than the first duration.

In some aspects, the techniques described herein relate to a method, further including providing a third drive signal to the transducer, the third drive signal having a third frequency different than the first frequency and the second frequency.

In some aspects, the techniques described herein relate to a patient interface, including: a connector, configured to connect the patient interface to a therapeutic ultrasound system; a transducer array, including a plurality of transducers, wherein the transducer array is in electrical communication with the connector; and a memory, configured to store information associated with each transducer of the transducer array, and wherein the memory is in electrical communication with the connector; and wherein the information associated with each transducer of the transducer array includes a position of each transducer with respect to the patient interface.

In some aspects, the techniques described herein relate to a patient interface, wherein the position of each transducer with respect to the patient interface indicates a range of target tissue thicknesses associated with each transducer of the transducer array.

In some aspects, the techniques described herein relate to a patient interface, wherein the memory is further configured to store an indicator of a patient anatomy type associated with the patient interface.

In some aspects, the techniques described herein relate to a patient interface, wherein the patient anatomy type is selected from the group consisting of: a foot, a calf, a thigh, a forearm, and a bicep.

### BRIEF DESCRIPTION OF THE DRAWINGS

Various features will now be described with reference to the following drawings. Throughout the drawings, reference numbers may be re-used to indicate correspondence between referenced elements. The drawings are provided to illustrate examples described herein and are not intended to limit the scope of the disclosure.

FIG. 1A illustrates a front perspective view of an example ultrasonic therapeutic device;

FIG. 1B illustrates a rear perspective view of the example ultrasonic therapeutic device of FIG. 1A;

FIG. 1C illustrates an example therapeutic system including a patient interface and ultrasonic therapeutic device;

FIG. 2 illustrates example components that may be included within the therapeutic system of FIG. 1C;

FIG. 3 is a plot of acoustic output of two transducers driven at the same input voltage;

FIG. 4A illustrates an example acoustic output measurement for a transducer driven at two different frequencies;

FIG. 4B illustrates the acoustic output of transducers driven at two different frequencies within a target site;

FIG. 5 is a flow diagram depicting an example routine for providing harmonized energy delivery from highly variable transducers in an array of an ultrasound-based sleeve.

FIG. 6 is a flow diagram depicting an example routine for delivering harmonized energy from highly variable transducers in an array based on stored output profiles

FIG. 7 is a flow diagram depicting an example routine for determining an optimal activation sequence for transducers in an array based on efficiency values.

FIG. 8 is a flow diagram depicting an example routine for activating transducers of an array based on an optimal activation sequence.

FIG. 9 is a flow diagram depicting an example routine for providing acoustic energy from transducers of an array at varying frequencies for varying desired therapy depths.

### DETAILED DESCRIPTION

Disclosed herein are systems and methods including a non-invasive ultrasound modalities tuned to deliver harmonized acoustic energy to target zones for treating a variety of medical conditions. The devices can be advantageously configured to achieve a variety of beneficial clinical effects, including but not limited to vasodilation and angiogenesis via collateralization and/or an increase in microvascular density.

Systems and its components, such as ultrasound-based sleeves or wraps, sometimes referred to as patient interfaces, may include a plurality of transducers configured to deliver acoustic energy to a target site. However, due to manufacturing limitations and anatomic variability, transducers of a TUS array may be preset to deliver variable energy levels. For example, the same drive voltage provided to two different transducers may result in different output energies from each transducer due to the inherent manufacturing differences. In order for both transducers to output the same energy level or provide peak negative pressure at a desired distance for an individual's anatomy, the circuitry or drive voltage of each transducer needs to be manually adjusted. Because an ultrasound-based sleeve may contain more than one transducer in an array, dissonant energy delivery may result if such adjustments are not made. Therefore, it would be advantageous to provide a profile corresponding to each transducer of the transducer array, each profile including a relationship between drive voltage and output energy so that the appropriate drive voltage may be provided to each individual transducer in order to improve output energy accuracy and consistency between transducers of the transducer array.

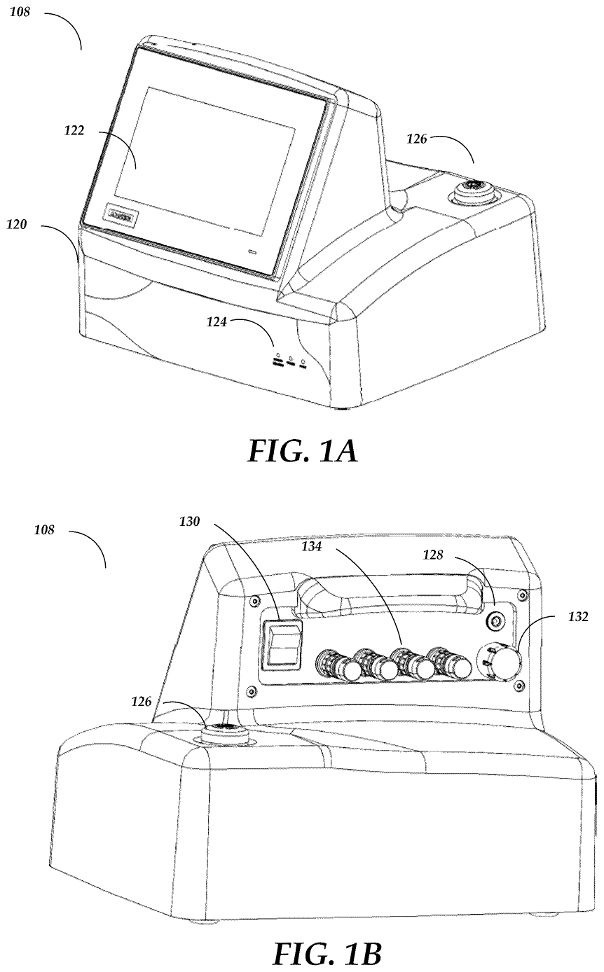

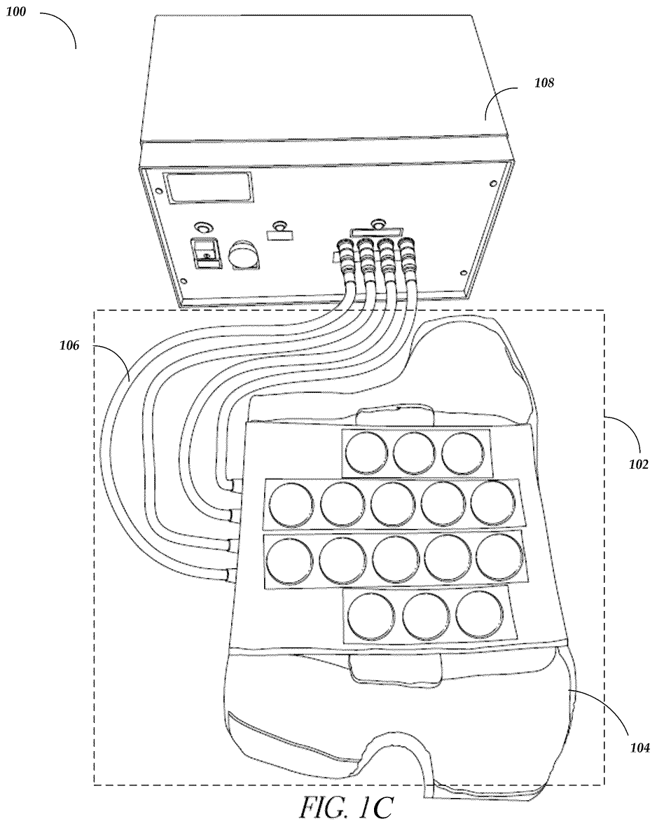

FIGS. 1A and 1B illustrate front and rear perspective views of one embodiment of an ultrasonic therapeutic device 108, sometime referred to as a therapeutic ultrasound device. The ultrasonic therapeutic device 108 includes a housing 120, which houses the internal components of the ultrasonic therapeutic device 108. A user interface 122 is supported by the housing 120 and is configured to provide user selectable controls and status information to a user. In one embodiment, the user interface 122 includes a touch screen. Various indicators 124, such as LEDs, may also be provided to indicate device 108 status. A stop switch 126 is also provided to enable immediate power deactivation, if necessary. For example, the stop switch 126, when activated, will cause the device 108 to immediately stop energy output by patient interface transducers, although the device 108 may remain powered on.

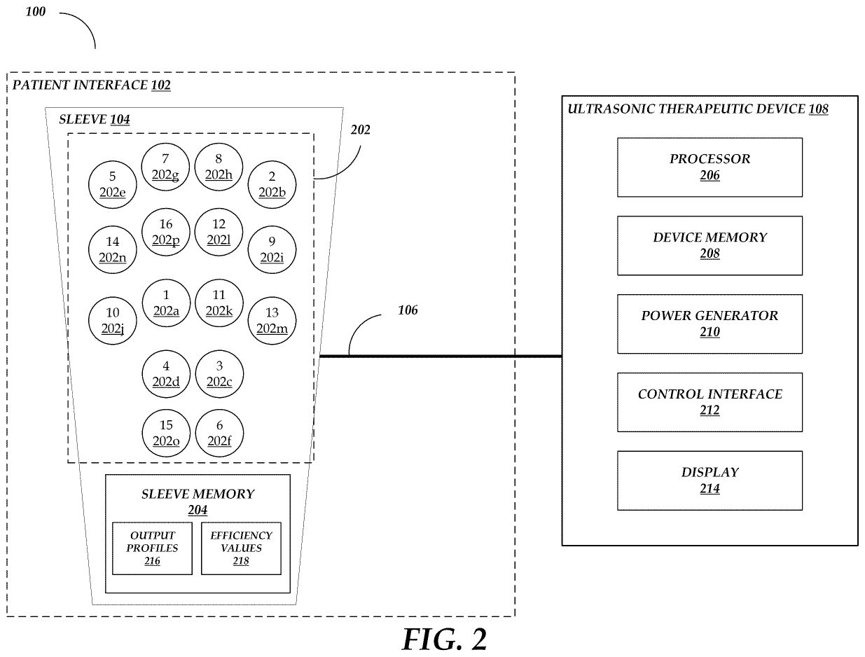

A power connector 128 at the rear of the housing 120 receives a power cord to provide electrical power to the device 108. A power switch 130 can switch power on and off to the device 108. A fuse holder 132 provides electrical protection to the device 108. Various patient interface cable connectors 134 allow the connection of a patient interface (not shown), including cables 106 of the patient interface 102 of FIGS. 1C and 2, to the device 108.

FIG. 1C illustrates therapeutic system 100 configured to deliver harmonized energy to a target site through a transducer array. As shown in FIG. 1C, therapeutic system 100 includes a patient interface 102 and an ultrasonic therapeutic device 108, sometimes referred to as a therapeutic ultrasound device. In some embodiments, patient interface 102 includes a sleeve 104 and one or more cables 106.

In some embodiments, a cable 106 may be configured to connect the sleeve 104 with the ultrasonic therapeutic device 108 and to transfer electrical energy, such as a drive voltage or a drive current (or both) to the individual transducers of the patient interface 102. In some embodiments, the cable 106 may include one or more cables or conductors, as shown in FIG. 1C.

In some embodiments, the patient interface 102 may include an ultrasound-based device for treating PAD or another disease. The device 100 can include a housing or component such as a sleeve 104 as shown, and/or a sock or other form factor, depending upon the patient limb or body structure being treated. In some embodiments, the housing such as a sleeve 104 can be configured to extend completely circumferentially around a body structure as shown, or only partially circumferentially or partially around a body structure. The sleeve 104 may optionally include a detachable section, such as a zipper, hook-and-loop fastener material, or the like, such as axially along a length of the sleeve 104, to improve ease of attachment or removal of the sleeve 104 to the patient. The sleeve 104 can include an elastic or inelastic material, or a combination.

FIG. 2 illustrates components that may be included in the therapeutic system 100. As shown in the embodiment of FIG. 2, the therapeutic system 100 includes patient interface 102 and an ultrasonic therapeutic device 108. The ultrasonic therapeutic device 108 includes a processor 206, a device memory 208, a power generator 210, a control interface 212, and a display 214, as discussed in further detail, below. The patient interface 102 includes a sleeve 104 and a cable 106, as discussed in further detail, below. The sleeve 104 includes a transducer array 202 and a sleeve memory 204, as discussed in further detail, below. The patient interface 102 may include the sleeve 104 and cable(s) 106, as described above with respect to in FIG. 1C.

In some embodiments, the patient interface 102 may include transducers configured to convert electrical energy into ultrasonic vibrations. Transducers, as used herein, may be configured to deliver acoustic energy (e.g., ultrasonic vibrations) at varying frequencies in response to varying input drive voltage frequencies. For example, transducers 202a-202N (shown in FIG. 2 as transducers 202-1 through 202-16) may deliver acoustic energy at any frequency between 400-800 kHz, including 400 kHz, 410 kHz, 420 kHz, 430 kHz, 440 kHz, 450 kHz, 460 kHz, 470 kHz, 480 kHz, 490 kHz, 500 kHz, 510 kHz, 520 kHz, 530 kHz, 540 kHz, 550 kHz, 560 kHz, 570 kHz, 580 kHz, 590 kHz, 600 kHz, 610 kHz, 620 kHz, 630 kHz, 640 kHz, 650 kHz, 660 kHz, 670 kHz, 680 kHz, 690 kHz, 700 kHz, 710 kHz, 720 kHz, 730 kHz, 740 kHz, 750 kHz, 760 kHz, 770 kHz, 780 kHz, 790 kHz, 800 kHz, or any other therapeutically effective frequency.

In some embodiments, the sleeve 104 may include a transducer array 202 including a plurality of transducers 202a, 202b, . . . 202N (e.g., 16 transducers, or other number) operably attached to an inner and/or outer surface of the sleeve. In some embodiments, one or more transducer 202a, 202b . . . 202N or the entire transducer array 202 can include flexible materials and can generally conform to the shape of the sleeve. In some embodiments, transducer array 202 may be configured to deliver ultrasonic energy to a target site via each transducer 202a-202N.

In some embodiments, the sleeve 104 may include sleeve memory 204. In some embodiments, the sleeve memory 204 may be configured to store information related to each transducer 202a-202N of the transducer array 202.

In some embodiments, sleeve memory 204 stores transducer output profiles (“output profiles”) 216. In some embodiments, the output profiles 216 characterize each transducer 202a-202N of the transducer array 202. For example, in some embodiments, the output profile 216 describes a relationship between a drive voltage applied to a particular transducer and an output power level produced by that transducer in response to the drive voltage. This characterization may occur as a step in the manufacturing process of the patient interface 102.

In order to characterize a single transducer, it can be connected to both a power generator and a power meter, multimeter, or any other relevant measuring device. The power generator may be configured to deliver a wide range of input voltages to the transducer to simulate output “power levels” that a user may select on the ultrasonic therapeutic device 108. In some embodiments, the output current level, power level, or other testing attributes resulting from the individual transducer may be recorded within the output profile 216 and stored in sleeve memory 204. Because the output profile for each individual transducer in transducer array 202 is likely to be slightly different, each transducer may require a different drive voltage in order to output a consistent power level across the transducer array 202. In some embodiments, sleeve memory 204 may store an output profile 216 that maps to each transducer of the transducer array 202.

In some embodiments, sleeve memory 204 may store more than one output profile 216 for each transducer. In some examples, output profiles 216 may be stored in sleeve memory 204 as a table, a database, or a formula, coefficients to a linear equation, a combination of the foregoing, etc. Accordingly, it is advantageous to store the output profiles 216 of each transducer 202a-202N of transducer array 202 to determine the correct drive voltage to assure consistent output power levels from the transducers 202a-202N of the transducer array 202. In addition, the storage of output profile 216 in the sleeve memory 204 (instead, for example, of hard coding the performance of the transducers within the ultrasonic therapeutic device 108), allows for use of patient interface 102 with any ultrasonic therapeutic device 108, as well as using any of a variety of patient interfaces 102 with an ultrasonic therapeutic device 108. This may be helpful in the case where a patient utilizes a single ultrasonic therapeutic device 108 to power additional sleeves, or when a sleeve 104 needs to be replaced.

In some embodiments, sleeve memory 204 may store transducer efficiency values 218, as well (or instead of output profiles). The storage of efficiency values 218 for each transducer of transducer array 202 may be used for energy conservation and to determine a beneficial transducer activation sequence. Each efficiency value 218 for each transducer may correspond to a ratio of transducer output power to input drive voltage. For example, an “efficient” transducer may be able to provide a higher output power at a given voltage, whereas a “less efficient” transducer would provide less output power at the same drive voltage. In some embodiments, this characterization may be determined using the output profiles 216 of each transducer. In some embodiments, the characterization of each transducer's efficiency values may be determined at the same step as the determination of the output profiles 216. In some embodiments, efficiency values may be determined by the ultrasonic therapy device 108 from the output profiles 216. In such embodiment, the efficiency values may or may not also be stored in the sleeve memory 204. To determine an efficiency value, for example, the ultrasonic therapeutic device may be configured to determine a best fit line or curve associated with each output profile and use a slope or other metric associated with such line or curve to determine an efficiency value associated with each transducer. In another embodiment, the efficiency value may be determined using transducer output profiles to determine an output power of each transducer at the particular drive voltage, or by determining a required drive voltage to achieve the same output power from each transducer. The sleeve memory 204 may or may not store efficiency values 218.

As shown in FIG. 2, ultrasonic therapeutic device 108 may be communicatively coupled to patient interface 102, for example, via a connector having one or more conductors and or cables 106. In some embodiments, ultrasonic therapeutic device 108 includes processor 206, device memory 208, power generator 210, control interface 212, and display 214.

In some embodiments, processor 206 may be configured to execute instructions stored in device memory 208 to perform various tasks within therapeutic system 100. For example, processor 206 may communicate with sleeve memory 204 via cable 106 in order to retrieve output profiles 216, efficiency values 218, or both. In some embodiments, a processor 206 may utilize output profiles 216 and efficiency values 218 to determine an appropriate input drive voltage for each transducer of the transducer array 202 when sleeve 104 is powered on. In some embodiments, the processor 206 may access efficiency values 218 to determine an optimal sequence in which to drive each transducer of the transducer array 202. In some embodiments, the processor 206 may be configured to receive and send signals between components of the therapeutic system 100. For example, in some embodiments, the processor 206 may receive information stored in the sleeve memory 204 related to a transducer array 202 supported by the sleeve 104. In some embodiments, the processor 206 may determine the input drive voltage for each transducer of transducer array 202 based on output profiles 216. In another embodiment, a control interface 212 may determine a sequence for driving the transducers of transducer array 202 based on the efficiency values 218.

In some embodiments, ultrasonic therapeutic device 108 may include a power generator 210. In some embodiments, the power generator 210 may be configured to provide a drive current and/or drive voltage to each transducer 202a-202N of the patient interface 102 in order to activate the transducer array 202. In some embodiments, the power generator 210 may provide the input drive voltage to power each transducer of the transducer array 202. As noted above, transducers of the transducer array 202 may be configured to convert electrical energy into acoustic energy in order to deliver ultrasonic therapy to a target site.

In some embodiments, control interface 212 may be configured to allow a user (patient, etc.) to operate the therapeutic system 100. In some embodiments, control interface 212 may include a console, buttons, keys, switches etc. In some embodiments, control interface 212 may be configured to allow a user to set a power level of energy to be delivered by the transducers 202a-202N of the transducer array 202 supported by the patient interface 102. In some embodiments, a “power level” of the patient interface 102 may refer to the amount of acoustic energy to be delivered to a target site. In some embodiments, control interface 212 may be configured to allow a user to control the frequency of the voltage signal delivered to the transducer array 202, which will correspond to the frequency of the acoustic signal emitted by each transducer 202a-202N of the transducer array 202.

In some embodiments, the control interface 212 allows a user to provide various patient parameter inputs which are used by a processor to determine an appropriate power level. The patient parameter inputs may include, for example, the patient's height, weight, body mass index (BMI), etc. In some cases, the control interface 212 includes a wired or wireless connection to a patient parameter measurement device. For example, the patient parameter measurement device can include a scale (to determine the patient's weight or mass), and/or a dimension measurement device. In one example, a band is wrapped around a patient's limb, and the circumference of the limb is automatically transferred to the control interface 212. The user may input the position of the band, as well. Band positions may correspond to treatment areas on the patient's limb. For example, the user may provide an input indicating that the band is positioned just below the knee, a particular distance below the knee (e.g., about 2″, about 4″, about 6″, about 8″, about 10″, about 12″, etc.), at the thickest part of the calf, and/or just above the ankle. In other cases, the user manually enters both the measurement information as well as the measurement position. A processor is configured to use the patient parameter inputs to determine or estimate the thickness of treatment tissue (e.g., the distance between the skin and the bone) at each transducer 202a-202N when the patient interface 102 is attached to the patient's limb, and to determine an appropriate power level for each respective transducer 202a-202N using the patient parameter inputs.

In some embodiments, ultrasonic therapeutic device 108 may include a display 214. In some embodiments, the display 214 may be configured to display a current state of the patient interface 102, such as an on/off status, a currently selected power level, a currently selected frequency, etc. In some embodiments, the control interface 212 may be integrated within display 214, such as via touchscreen, etc.

In some examples, an output profile 216 may be used to deliver harmonized energy to a target site. For example, in some embodiments, the patient interface 102 may be connected to an ultrasonic therapeutic device 108 in order for a patient to utilize and receive ultrasound therapy via the patient interface 102. As noted above, the sleeve memory 204 may contain output profiles 216 and efficiency values 218 for each transducer of the transducer array 202. In some embodiments, the processor 206 may be configured to access information stored in sleeve memory 204, such as the output profiles, efficiency values 218, etc. In addition, the processor 206 may be configured to access information from the control interface 212. In some embodiments, the processor 206 may utilize information from both the control interface 212, such as a desired power level (e.g., an input value entered by the user), and output profiles 216 in order to determine an appropriate drive voltage for each transducer of transducer array 202. Accordingly, the processor 206 may send a signal to the patient interface 102 to drive each transducer at the determined applied drive voltage such that the output acoustic power is consistent across the transducer array 202.

It is noted that power generator 210 provides current to each transducer of transducer array 202. In some embodiments, power generator 210 may activate each transducer one at a time (e.g., drive a first transducer before moving to a second transducer until all have been activated). Because efficiency values 218 may be different for each transducer of the transducer array 202, a random charge sequence may lead to excess voltage loss. Accordingly, charging the transducers from most efficient (utilizing the lowest drive voltage or drive current) to least efficient (utilizing the highest drive voltage or drive current) may lead to optimal use of input voltage with less loss between subsequent drive signals. In addition to providing harmonized output, therapeutic system 100 may be configured to drive the transducer array 202 based on efficiency values 218. As noted above, the sleeve memory 204 may contain output profiles 216 and efficiency values 218 for each transducer of transducer array 202. In some embodiments, processor 206 may be configured to access information stored in the sleeve memory 204. In addition, the processor 206 may be configured to access information from the control interface 212. In some embodiments, the processor 206 may utilize information from both the control interface 212, such as a desired power level (e.g., an input value entered by the user), and output profiles 218 in order to determine an optimal sequence in which to drive each transducer of the transducer array 202 in order to conserve energy or in order to assure that the drive voltage will only be increased or maintained at the previous level (and not decreased) when stepping through the transducer 202a-202N activation sequence. In some examples, the optimal sequence may order the transducers from most efficient (driving the transducer at the lowest voltage or current) to least efficient (driving the transducer at the highest voltage or current). As shown in FIG. 1C, numbers 1-16 on each transducer of transducer array 202 represent an optimal sequence ranking each transducer based on efficiency. In some embodiments, this sequence may be stored in sleeve memory 204. Accordingly, processor 206 may send a signal to patient interface 102 to activate each transducer of transducer array 202 in the optimal sequence.

Each treatment (e.g., the power delivered, the duration of delivery, the frequency of the delivered energy, etc.) delivered by each transducer of the transducer array 202 may be stored by the ultrasound device in a memory, and may be viewed and/or retrieved by an operator at a later time via a wired or wireless connection (e.g., via USB port or 802.11 transmission, such as WI-FI® or BLUETOOTH®).

In some examples, therapeutic system 100 may deliver harmonized energy to a target site having a frequency or within at a range of frequencies.

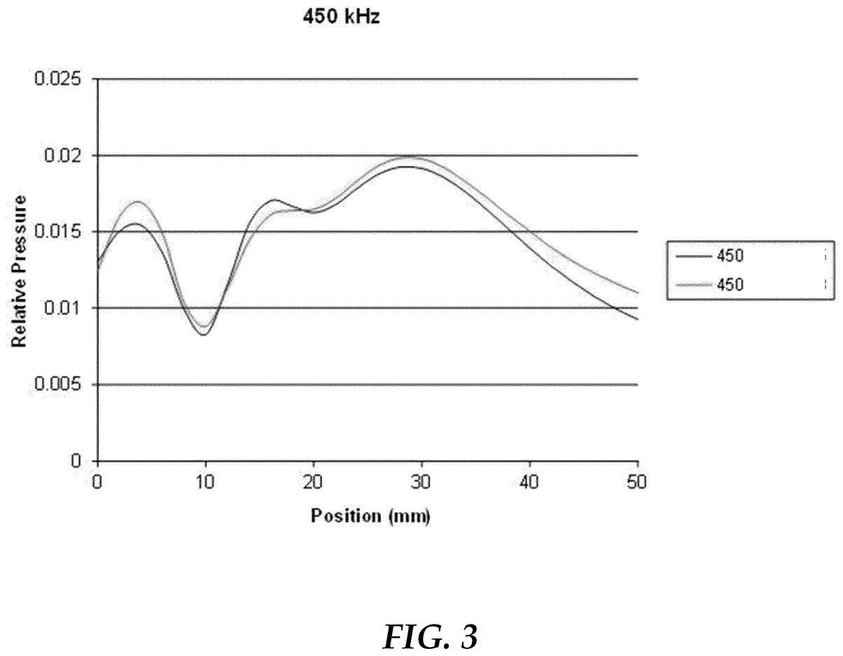

FIG. 3 is a plot of acoustic outputs of two transducers driven at the same input, drive voltage. As shown in FIG. 3, the y-axis indicates the relative pressure (measured with a pressure transducer in millivolts) of the transducers, while the x-axis indicates the depth of the acoustic energy (in mm) from transducer array 202 into the target site. As shown, the acoustic outputs between the two transducers may vary slightly although both are driven at the same input, drive voltage. For example, at approximately 4 mm from each transducer's face, each transducer provides a relative peak pressure (corresponding to a peak in energy output); however, the relative peak pressure of the SN08 transducer is approximately 0.017 mV while the relative peak pressure of the SN05 transducer is only about 0.0155 mV. Similarly, at approximately 29 mm from each transducer's face, each transducer provides another relative peak pressure. The relative peak pressure of the SN08 transducer is again higher than that of the SN05 transducer. Therefore, to achieve the same pressure from both transducers SN05 and SN08 at the same distance from each sensor's face, the SN05 transducer (which is less efficient) would be driven at a slightly higher voltage or current than the SN08 transducer (which is more efficient), or the SN08 transducer (which is more efficient) would be driven at a slightly lower voltage or current than the SN05 transducer (which is less efficient). This characterization may be used in the tuning of each individual transducers according to the methods described in FIG. 2.

In another embodiment, the drive voltage to each transducer is gradually increased, or ramped up, over a predetermined period. For example, over a period of 3 min, 5 min, 7 min, 10 min, 15 min or 30 min, the drive voltage may be slowly increased from 0% to 100% of the maximum drive voltage for a particular power setting. Ramping drive voltage can provide a more comfortable experience to the patient and allows the patient to become less sensitive to the ultrasonic therapy.

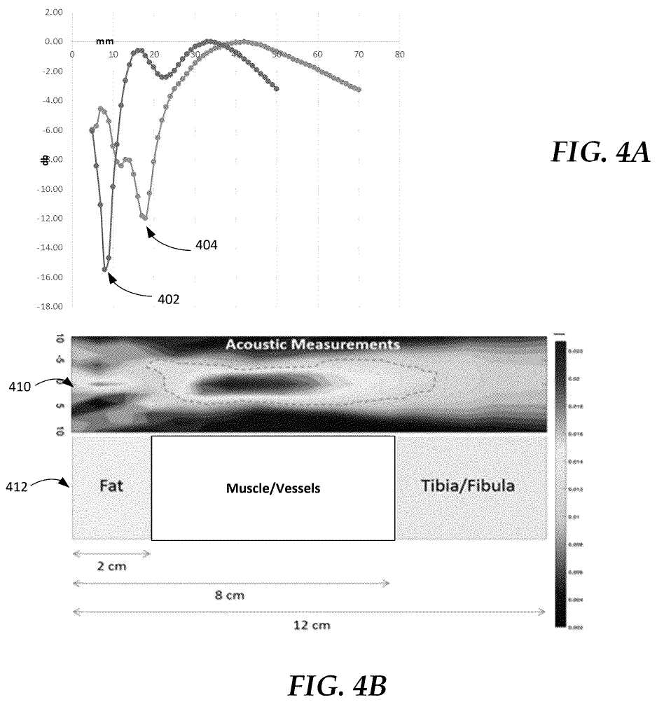

FIG. 4A illustrates an example acoustic output measurement of a transducer driven at two different frequencies. As shown in FIG. 4A, the y-axis indicates the output intensity of the transducer (e.g., any one of transducer 202a-202N) measured in decibels (dB) (which is a measure of acoustic output energy or pressure from the transducer) and the x-axis indicates the depth of the acoustic energy (in mm) from transducer output surface within a target site, such as a patient's calf. As shown, first ultrasonic signal 402 may have a first frequency. In one embodiment, the first ultrasonic signal 402 has a frequency of 450 kHz. As first ultrasonic signal 402 moves along the x-axis (e.g., travels deeper into the body), first ultrasonic signal 402 displays a gain between-2 dB and 0 dB between the depth of 12 mm to 45 mm. In some embodiments, a gain between-2 dB and 0 dB is sufficient to deliver therapeutically effective ultrasonic energy to a target site. Accordingly, first ultrasonic signal 402 may be configured to deliver ultrasonic energy to a target site at a depth of 12 mm to 45 mm.

Also as shown, second ultrasonic signal 404 may operate at a second frequency. In one embodiment, the second ultrasonic signal 404 has a frequency of 500 KHz. As second ultrasonic signal 404 moves along the x-axis (e.g., travels deeper into the body), first ultrasonic signal 404 displays a gain between-2 dB and 0 dB between the depth of 30 mm to 60 mm.

FIG. 4B illustrates the acoustic output of transducers driven at two different frequencies within a target site. It is noted that the first ultrasonic signal 402 and the second ultrasonic signal 404 may correspond to graphical output 410 of FIG. 4B. As shown in FIG. 4B, the peaks of first ultrasonic signal 402 and second ultrasonic signal 404 correspond to the treatment zone 412 of FIG. 4B. Accordingly, patient interface 102 may be configured to deliver acoustic energy at varying frequencies in order to target a range of depths within a patient's body, such as within the muscle/vessels. Thus, if the target treatment area is relatively large, utilizing multiple frequencies for the same transducer will provide changes in distances from tissue surface to the location peak negative pressure, allowing for treatment of a larger area than a constant, non-alternating (or single) frequency.

In some embodiments, the ultrasonic therapeutic device 108 may cycle between the two drive frequencies to ensure delivery of a therapeutically effective amount of ultrasonic energy within a desired volume of tissue. For example, by switching between the two drive frequencies of FIG. 4A, therapeutic ultrasonic energy would be delivered about 12 to 45 mm during at the first drive frequency and 30 to 60 mm during the second drive frequency resulting in an effective treatment area of 12 to 60 mm deep into the target tissue. In other embodiments, the number of frequencies at which each sensor is driven varies based upon the position of the sensor in the sensor array 202, and the thickness of the target tissue against which each such sensor is positioned. This allows to achieve the desired distance for peak pressure that corresponds to a particular patient's anatomy. Thus, the same transducer could be driven at different frequency from one patient to another to better correspond to the desired target based on patient's anatomy and transducer placement relative to the target.

For example, in treatment of PAD, in some cases it may be undesirable to deliver ultrasonic energy into bone tissue. Therefore, if the thickness of the muscle and fat between a particular transducer output surface and bone is less than about 50 mm (e.g., at the lower calf muscle), then then transducer adjacent such muscle and fat may be driven at only one frequency (e.g., 450 kHz, corresponding to the first ultrasonic signal 402, discussed above). However, if the thickness of the muscle and fat between the transducer output surface and bone is greater than 50 mm and at least about 65 mm (e.g., at the mid to upper calf muscle), then ultrasound transducer may be driven at both frequencies to achieve both output signals 402, 404.

For example, the transducer may be initially activated at the first drive frequency (e.g., 450 kHz) for a first period of time and then activated at the second drive frequency (e.g., 500 kHz) for a second period of time. This cycle may be repeated as many times as desired. In one embodiment, the system may be configured to receive an input corresponding to a patient's calf measurement circumference at different distances from a reference location (e.g., distance above the ankle, distance below the knee, distance from bottom of calf muscle, distance from top of calf muscle, etc.), and the system may automatically determine whether transducers to be positioned at such distances should be activated with one, two, or more drive frequencies.

In another embodiment, the system includes different calf profiles (e.g., corresponding to different sized calves) and a user can select a profile that best matches the calf size of the patient. The system can then determine whether each transducer of the transducer array 202 should be driven at one, two, or more drive frequencies.

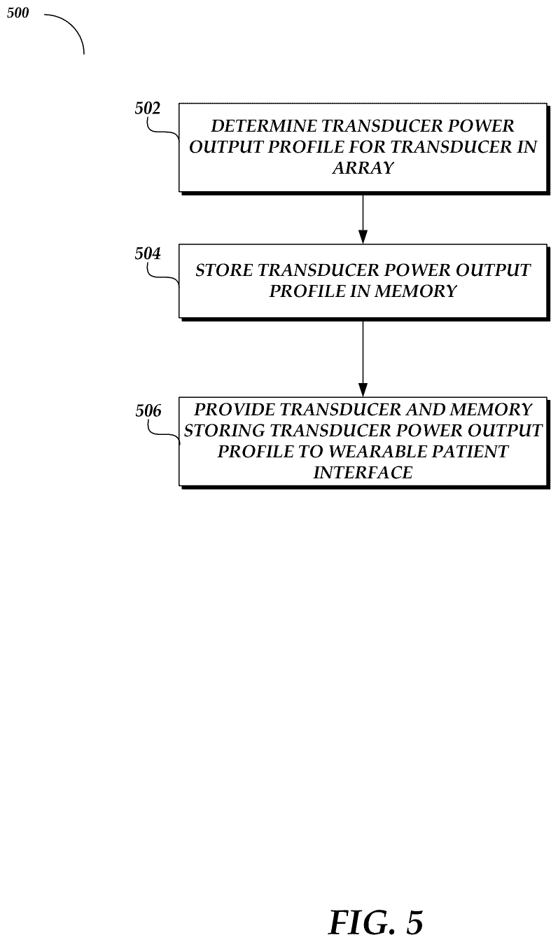

FIG. 5 is a flow diagram depicting an example routine for providing harmonized energy delivery from highly variable transducers in an array of an ultrasound-based sleeve. In some embodiments, routine 500 may be implemented by a processor 206 of therapeutic system 100. It is noted that routine 500 may be implemented in combination with any other routine provided herein.

At block 502, the processor determines, for a transducer of a transducer array, a transducer power output profile. In some embodiments, the transducer power output profile describes a relationship between a drive voltage applied to the transducer and an output power level produced by the transducer in response to the drive voltage.

In some embodiments, wherein the transducer power output profile includes a table, a lookup table, a database, or a formula.

In some embodiments, the transducer power output profile describes a relationship between the drive voltage applied to the transducer at a frequency and an output power level produced by the transducer in response to the drive voltage.

In some embodiments, the transducer power output profile describes a relationship between a range of drive voltages applied to the transducer and output power levels produced by the transducer in response to the range of drive voltages.

In some embodiments, the transducer power output profile describes a relationship between the drive voltage applied to the transducer at a frequency and an output power level produced by the transducer in response to the drive voltage.

At block 504, the processor stores the transducer power output profile in a memory of a patient interface.

At block 506, the processor provides, from the memory, the transducer power output profile to the patient interface.

In some embodiments, routine 500 further comprises determining, for the transducer of the transducer array, a transducer depth profile. In some embodiments, the transducer depth profile describes a relationship between a drive frequency applied to the transducer and a depth of penetration achieved by the transducer in response to the drive frequency.

In some embodiments, routine 500 further comprises determining an applied drive voltage for the transducer based upon the transducer power output profile and a user input. In some embodiments, the user input includes a desired power level. In other embodiments, the user input is not limited to a desired power level, but may include other values that correspond to, or may be further processed to determine, a desired or appropriate output power level. For example, a distance to a target, a patient's calf size or other anatomical mark or parameter(s) (e.g., height, weight, body-mass index value, any other anatomical mark or a combination of such marks) may be used by the system to determine a desired output power level. In other embodiments, a closed-loop system is provided that includes sensors to measure or detect such parameter(s) and automatically determine a desired power level based on such closed-loop measurements and feedback.

In some embodiments, routine 500 further comprises driving the transducer at the desired power level.

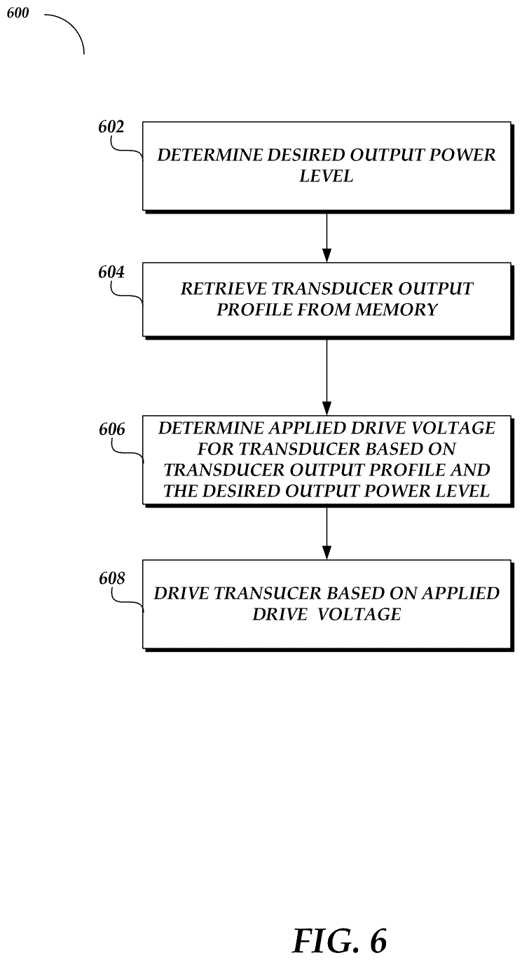

FIG. 6 is a flow diagram depicting an example routine for delivering harmonized energy from highly variable transducers in an array based on stored output profiles. In some embodiments, routine 600 may be implemented by a processor 206 of therapeutic system 100. It is noted that routine 600 may be implemented in combination with any other routine provided herein.

At block 602, the processor determines a desired output power level. For example, the processor may receive a user input corresponding to a desired output power level or it may calculate a desired output power level based upon a therapeutic treatment protocol.

At block 604, the processor retrieves, for a transducer of a transducer array, a transducer power output profile. In some embodiments, the transducer power output profile describes a relationship between a drive voltage applied to the transducer and an output power level produced by the transducer in response to the drive voltage. In some embodiments, the transducer power output profile includes a table, a lookup table, a database, or a formula. In some embodiments, the transducer power output profile describes a relationship between the drive voltage applied to the transducer at a frequency and an output power level produced by the transducer in response to the drive voltage.

At block 606, the processor determines an applied drive voltage for the transducer based upon the transducer power output profile and the desired output power level. In some embodiments, processor may further determine a plurality of applied drive voltages for the transducer based upon the transducer power output profile and a user input.

At block 608, the processor drives the transducer of the transducer array at the applied drive voltage.

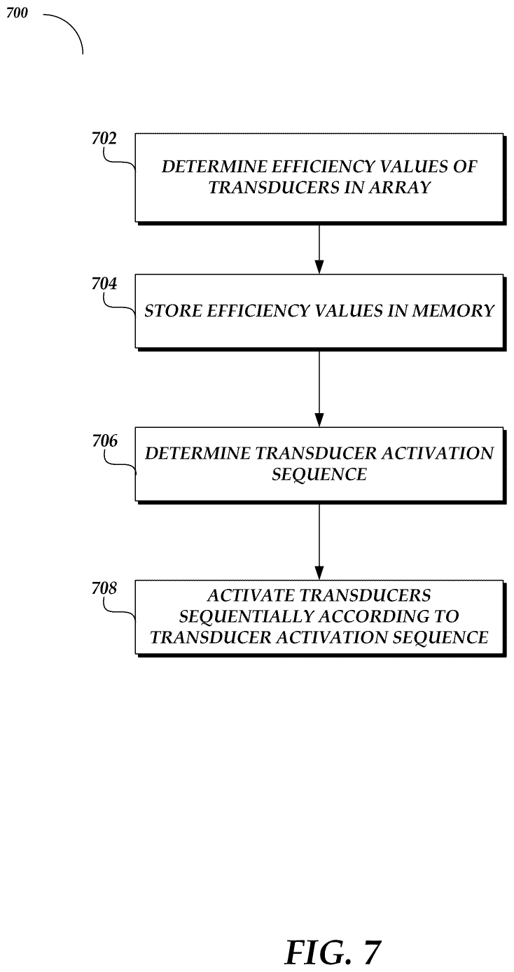

FIG. 7 is a flow diagram depicting an example routine for determining an optimal activation sequence for transducers in an array based on efficiency values. In some embodiments, routine 700 may be implemented by a processor 206 of therapeutic system 100. It is noted that routine 700 may be implemented in combination with any other routine provided herein.

At block 702, the processor determines, for each transducer of a transducer array, an efficiency value. In some embodiments, the efficiency value corresponds to a relationship between an output power of each transducer at a particular input drive voltage applied.

At block 704, the processor stores the efficiency value for each transducer of the transducer array in a memory of a patient interface.

At block 706, the processor determines a transducer activation sequence based upon the efficiency value of each transducer of the transducer array. In some embodiments, the transducer activation sequence is based upon the efficiency value of each transducer of the transducer array from a highest efficiency value to a lowest efficiency value. In some embodiments, the transducer activation sequence is based upon the efficiency value of each transducer of the transducer array from a lowest efficiency value to a highest efficiency value. In some embodiments, the processor is further configured to store the transducer activation sequence in the memory of the patient interface.

At block 708, the processor activates the transducers sequentially according to the transducer activation sequence. In some embodiments, activating comprises delivering, by each transducer, a number of pulses at a specified frequency and specified duration. In some embodiments, activation of each transducer occurs during a ramp-up period. In some embodiments, the transducer array is housed within the patient interface.

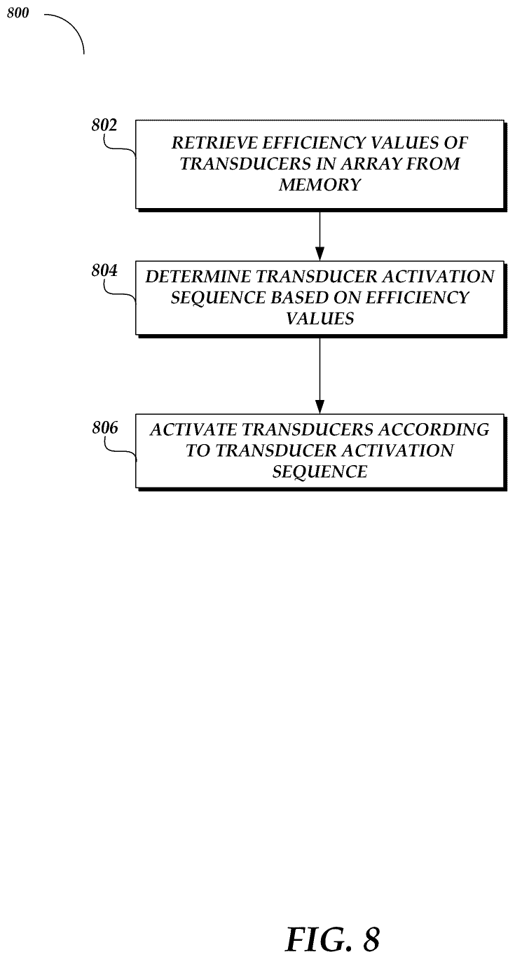

FIG. 8 is a flow diagram depicting an example routine for activating transducers of an array based on an optimal activation sequence. In some embodiments, routine 800 may be implemented by a processor 206 of therapeutic system 100. It is noted that routine 800 may be implemented in combination with any other routine provided herein.

At block 802, the processor retrieves, for each transducer of a transducer array, an efficiency value stored in a memory of a patient interface. In some embodiments, the efficiency value corresponds to a relationship between an output power of each transducer at a particular input drive voltage applied.

At block 804, the processor determines a transducer activation sequence based upon the efficiency value of each transducer of the transducer array. In some embodiments, the transducer activation sequence is based upon the efficiency value of each transducer of the transducer array from a highest efficiency value to a lowest efficiency value.

At block 806, the processor activates each transducer of the transducer array according to the transducer activation sequence. In some embodiments, activating comprises delivering, by each transducer, a number of pulses at a specified frequency and specified duration. In some embodiments, activation of each transducer occurs during a ramp-up period.

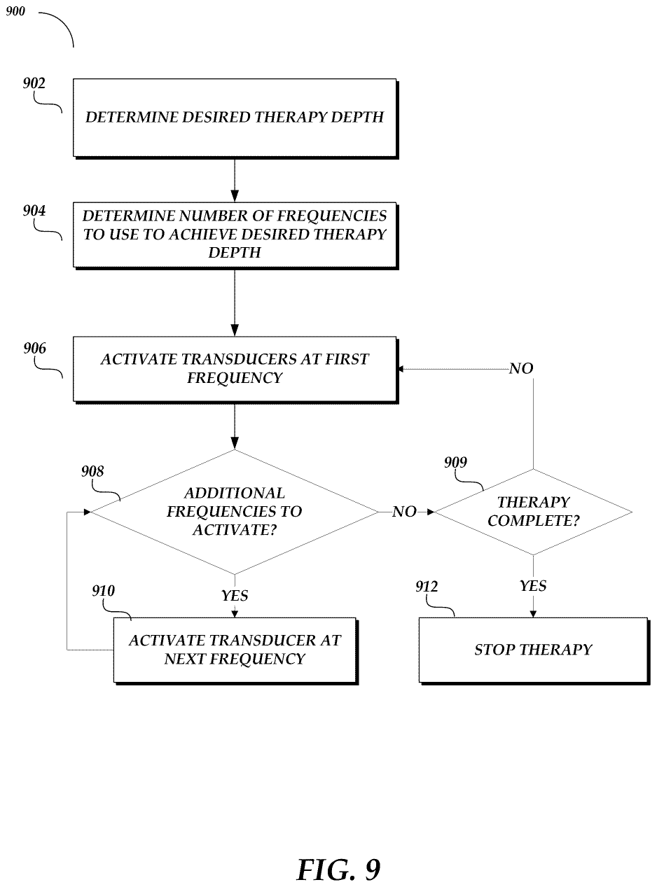

FIG. 9 is a flow diagram depicting an example routine for providing acoustic energy from transducers of an array at varying frequencies for varying desired therapy depths. In some embodiments, routine 900 may be implemented by a processor 206 of therapeutic system 100. It is noted that routine 900 may be implemented in combination with any other routine provided herein.

At block 902, the processor may determine a desired therapy depth or depths. In some embodiments, the desired therapy depth may be specified by a user via control interface 212. In some embodiments, the desired therapy depth may range from 0-10 cm, such as 1 cm, 2 cm, 3 cm, 4 cm, 5 cm, 6 cm, 7 cm, 8 cm, 9 cm, or any depth in between said range, etc. In some embodiments, the desired therapy depth may correspond to a particular patient's leg profile and may correspond to a depth of muscles and/or vessels of the particular user.

At block 904, the processor may determine a number of frequencies to use in order to achieve the desired therapy depth or depths. As noted above, therapeutic system 100 may be used to target a range of depths depending on the frequency (see FIGS. 4A and 4B). Accordingly, in some embodiments, the processor may determine multiple frequencies to cycle through in order for therapeutic system 100 to target said range of depths.

At block 906, the processor may activate the transducers of transducer array 202 at a first frequency. In some embodiments, the transducers activated a first frequency may target a first depth.

At block 908, the processor may determine whether there are any additional frequencies to activate.

If there are additional frequencies to target, the processor may, at block 910, activate the transducers of transducer array 202 at a next frequency in order to target an additional depth.

If there are no additional frequencies to target, the processor may then determine if therapy is complete at block 909. For example, the total duration of a therapy session may include several applications of energy at the one or different transducer frequencies. Therefore, at block 909 if therapy has been completed, the method 900 may then continue to block 912, where the processor may stop therapy. If therapy is not complete, the method 900 may return to block 906.

### Device Specifications

Conventional ultrasound-based treatments for both CAD and PAD have been largely limited by the fact that treatment requires a healthcare provider to be present to position and hold the device. By providing a TUS sleeve that positions and fixes the ultrasound transducer or transducer array in place, treatment may be provided for up to several hours (e.g., 20 min-24 hours duration or more, including overnight therapy) at a time, thereby increasing treatment duration. Providing extended treatment duration using ultrasound-based devices at predetermined parameters can in some embodiments can lead to profound and unexpected improvements in therapeutic results, including but not limited to increased blood flow from, for example, vasodilation or angiogenesis (forming new blood vessels). FIGS. 1 and 2 illustrates potential mechanisms of action of, for example, TUS therapy. Not to be limited by theory, such a device can be configured to increase vascular permeability from cavitation microbubbles interacting with the endothelium, and shear stresses from ultrasound waves directly onto endothelial surfaces, which can stimulate the production and/or release of growth factors, angiogenic factors and signaling molecules such as increase tissue vascular endothelial growth factor (VEGF), endothelial nitric oxide synthase (eNOS), basic fibroblast growth factor (bFGF), adenosine triphosphate (ATP), for example, leading to angiogenesis and/or collateralogenesis. Longer duration treatments can advantageously increase the local and possibly also circulating levels of these angiogenic factors among others, leading to collateralogenesis and increased microvascular density in PAD.

Not to be limited by theory, long-term angiogenesis, vasodilation, and/or collateralogenesis can occur, for example, through two or more ultrasound-mediated mechanisms. The first mechanism is cavitation: in some embodiments, TUS waves with sufficient peak negative pressure may cause dissolved gas to come out of solution in blood and tissue, and to convert into microbubbles. In response to TUS, these bubbles then volumetrically oscillate and/or burst, interacting with vascular endothelial cells, increasing vascular permeability, and triggering angiogenesis, vasodilation, and/or collateralogenesis. While the process of cavitation is well-described, the inventors are not aware of previous techniques which specifically harness this process to promote vascular permeability and thus angiogenesis, vasodilation, and/or collateralogenesis. This mechanism may also trigger up-regulation of several molecular mediators of angiogenesis, vasodilation, and/or collateralogenesis as described further herein. In some embodiments, p− can be selected to promote cavitation, vascular permeability and angiogenesis, vasodilation, and/or collateralogenesis without leading to harmful or lethal vascular damage.

A second mechanism is shear stress: in some embodiments, TUS waves of a desired frequency and sufficient amplitude can directly interact with endothelial cells, triggering shear stress signaling pathways, which may lead to angiogenesis, vasodilation, and/or collateralogenesis. While the effects of endothelial shear stress on vasodilation and angiogenesis have been described, the inventors are not aware of previous techniques utilizing TUS to specifically increase endothelial shear stress, leading to vasodilation, collateralogenesis and/or angiogenesis.

TUS and SWT can in some embodiments lead to tissue-specific increases in angiogenic factors (or upregulation of receptors of growth factors) such as vascular endothelial growth factor (VEGF), e.g., VEGF-A and its receptor, FLT-1; fibroblast growth factor (FGF), e.g., bFGF the nitric oxide pathway; and stem cell differentiation. Systems and methods as disclosed herein can also potentially modulate (e.g., decrease or increase depending on the factor) levels of other factors including but not limited to VEGFR, bFGF, HIF-1-alpha, Egln1, NRP-1, Ang1, Ang2, PDGF, PDGFR, TGF-beta, endoglin, CCL2, ephrin, histamine, integrins, plasminogen activators, plasminogen activator inhibitor-1, eNOS, iNOS, COX-2, AC133, ID1/ID3, or class 3 semaphorins, among others. In some embodiments, the ultrasound-based therapy can change, such as increase or decrease circulating levels, mRNA, or other proxies of the foregoing markers by about or at least about 5%, 10%, 15%, 20%, 25%, 30%, 35%, 40%, 45%, 50%, 75%, 100%, or more after therapy compared to pre-therapy values. In some embodiments, blood flow at a desired target location can increase by about or at least about 5%, 10%, 15%, 20%, 25%, 30%, 35%, 40%, 45%, 50%, 75%, 100%, or more after therapy compared to pre-therapy values, and remain increased for about or at least about 6 hours, 12 hours, 1 day, 2 days, 3 days, 4 days, 5 days, 6 days, 1 week, 2 weeks, 1 month, 3 months, 6 months, 1 year, or even more. In some embodiments, the ultrasonic energy can be therapeutically effective to provide anti-inflammatory effects, stem cell differentiation, satellite cell differentiation, and/or modulation of prostacyclin pathways.

Endothelial cells line mature blood vessels and typically do not proliferate. However, if endothelial cells are activated by an angiogenic growth factor, they can proliferate and migrate into un-vascularized tissue to form new blood vessels. Blood vessels are surrounded by biological tissue in an extracellular matrix. The formation of new blood vessels is a function of the interactions between endothelial cells and the interaction of the endothelial cells with the extracellular matrix. These interactions are regulated by receptors on the surface of endothelial cells, which are sensitive to particular molecules such as angiogenic growth factors. Shear stress induced on endothelial cells by pressure waves can potentially reduce endothelial dysfunction and promotes angiogenesis and/or vasodilation. This effect can correlate in some cases with both with TUS amplitude (p⁻), as well as frequency (with greater shear stress at lower frequencies). Sub-lethal microvascular permeability can result from the process of “cavitation”: the formation and subsequent violent vibration/collapse of gas bubbles coming out of solution in vessels and interacting with the vessel wall via multiple mechanisms. This can be in some cases a threshold-based phenomenon, which occurs at a given p⁻ and increases with greater intensity. In other words, angiogenesis and/or vasodilation can be caused by stress/cavitation leading to endothelial signaling, growth factor increase, and new capillary and large vessel growth.

In some embodiments, an ultrasound-based device can be worn and operated for about or at least about 5, 10, 15, 20, 30, 40, 50, or 60 minutes daily, or about or at least about 2, 3, 4, 5, 6, 7, 8, 9, 10, 11, 12, 15, 18, 24, or more hours at a time (or ranges including any two of the aforementioned values), either cumulatively in multiple treatment sessions, or continuously in some cases. In some embodiments, the device can be worn and operated for between about 10 minutes and about 20 minutes; between about 20 minutes and about 40 minutes; between about 30 minutes and about 60 minutes; between about 1 hour and about 2 hours; between about 2 hours and about 4 hours; or between about 4 hours and about 8 hours per treatment session. However, in some embodiments the device is worn and operated for about, or no more than about 24, 18, 15, 12, 11, 10, 9, 8, 7, 6, 5, 4, 3, or 2 hours at a time. In some embodiments, the device can be worn and operated about or at least about once, twice, three times, or more daily; or once, twice, or three times weekly. In some embodiments, the device can be worn and operated overnight and/or while a patient is sleeping, such as between about 4 hours and about 10 hours, or between about 5 hours and about 9 hours daily or nightly, 5-7 times a week, or during the day while not sleeping.

In some embodiments, the devices allow for convenient dose-response titrations to readily be performed without requiring long treatments to be performed by a medical provider using timeframes such as disclosed above.

In some embodiments, the ultrasound modality could be TUS, SWT, or a dual-mode combination thereof using one or a plurality of ultrasound transducers. In some embodiments, use of TUS (which may include HIFU, LIPUS, or other pulsed or continuous wave acoustic energies) instead of SWT can advantageously allow for titration of one, two, or more acoustic parameters to achieve a desired angiogenic effect as discussed herein. The parameters can include, for example, frequency, pulse repetition frequency (PRF), pulse duration, duty factor, and pressure amplitudes (peak positive and negative pressures; p⁺, p⁻). Additionally, in some cases TUS can be advantageous as it allows application of multiple sound/pressure waves in each pulse; SWT provides a single pressure wave.

Due to differences in the SWT and TUS waveforms, SWT parameters can only be adjusted to modulate pulse repetition frequency and acoustic amplitudes. In contrast, TUS additionally allows modulation of ultrasound frequency, pulse duration, duty factor), parameters may be titrated to improve these angiogenic effects (low frequency, high p⁻), while avoiding acoustic intensities that may lead to thermal or cavitation-based damage. However, embodiments can also include SWT, including parameters for pulse repetition frequency and amplitudes as described herein. Furthermore, if regulatory requirements specify a maximal acoustic intensity (p⁻, W or W/cm²) to avoid cavitation-based damage, this parameter can be fixed while others can be adjusted to maximize effect. Finally, adverse effects of ultrasound are most prominent in gas-filled organs such as the lung and gastrointestinal tract in which gas unpredictably reflects and may intensify sound waves. Targeting lower extremity muscle and vasculature, which are generally free of air, can advantageously avoid these effects in some cases.

The above-described potential mechanisms of TUS-induced cavitation and shear stress can be dependent upon p− and frequency, respectively, although total dose of TUS is also determined by pulse repetition frequency (PRF), duty factor (% of time that TUS is active), and duration of therapy (time that patient wears the sleeve). Each of these TUS parameters has a toxic-therapeutic window, which can advantageously be adjusted for a desired clinical result given its design and titratability of TUS parameters.

Many of the TUS mechanisms promoting angiogenesis, vasodilation, and/or collateralogenesis with long-term use are also associated with acute, short-term vasodilation. Thus, certain embodiments of the device and method may be used to immediately or quickly increase perfusion for the treatment of acute limb ischemia, such as about or within about 1 minute, 5 minutes, 10 minutes, 15 minutes, 30 minutes, 60 minutes, 2 hours, 4 hours, 6 hours, 12 hours, 18 hours, or 24 hours after the onset of therapy, as well as have longer-lasting effects as disclosed herein. In some embodiments, systems and methods as disclosed herein can include only TUS and not SWT, only SWT but not TUS, or a combination of both SWT and TUS.

### Frequency

In some embodiments, the frequency of ultrasound provided could be between about 250 kHz and about 3 MHz, between about 250 kHz and about 1 MHz, between about 250 kHz and about 500 kHz, between about 1 MHz and about 3 MHz, between about 750 kHz and about 1.25 MHz, between about 500 kHz and about 1 MHz, between about 2 MHz and about 3 MHz, or overlapping ranges thereof. Not to be limited by theory, lower frequencies can advantageously increase the shear stress mechanism of action. In some embodiments, lower frequencies could also penetrate more deeply into issue, although frequencies that are too low may penetrate too deeply and reach bone on the opposite end of the desired PAD field. In some embodiments, the treatment frequency could be between about 250 kHz and about 1 MHz on the thigh (deeper field from the skin of the medial thigh to the femur); between about 500 kHz and about 1.25 MHz on the calf (deep field from the skin of the posterior calf to the tibia); or between about 750 kHz and about 1.5 MHz on the ankle (shallow field from the skin of the anterior ankle to the bones), and between about 1 MHz and about 3 MHz on the plantar surface of the foot (even shallower field from skin to the tarsal and metatarsal bones), or between about 500 kHz and about 3 MHz, between about 1 MHz and about 3 MHz, and/or at least about 500 kHz or 1 MHz in any of the aforementioned locations. In some embodiments, the frequency provided can be about, more than about, or no more than about 200 kHz, 250 kHz, 300 kHz, 350 kHz, 400 kHz, 450 kHz, 500 kHz, 550 kHz, 600 kHz, 650 kHz, 700 kHz, 750 kHz, 800 kHz, 850 kHz, 900 kHz, 950 kHz, 1 MHz, 1.1 MHz, 1.2 MHz, 1.3 MHz, 1.4 MHz, 1.5 MHz, 1.6 MHz, 1.7 MHz, 1.8 MHz, 1.9 MHz, 2 MHz, 2.1 MHz, 2.2 MHz, 2.3 MHz, 2.4 MHz, 2.5 MHz, 2.6 MHz, 2.7 MHz, 2.8 MHz, 2.9 MHz, 3 MHz, 3.1 MHz, 3.2 MHz, 3.3 MHz, 3.4 MHz, 3.5 MHz, 4 MHz, 5 MHz, 10 MHz, 15 MHz, 20 MHz, 25 MHz, 30 MHz, 35 MHz, 40 MHz, 45 MHz, 50 MHz, 60 MHz, 70 MHz, 80 MHz, 90 MHz, 100 MHz, or ranges incorporating any of the foregoing values. In some embodiments, systems and methods can provide a plurality of different alternating frequencies during treatment, such as 2, 3, 4, or more different frequencies.

### Pulse Repetition Frequency

In some embodiments, the pulse repetition frequency can be between about 0.1 Hz and about 100 Hz, between about 1 Hz and about 3 Hz, between about 0.1 Hz and about 1 Hz, between about 0.5 Hz and about 2 Hz, between about 1 Hz and about 5 Hz, between about 5 Hz and about 10 Hz, between about 10 Hz and about 20 Hz, between about 20 Hz and about 100 Hz, or overlapping ranges thereof. Not to be limited by theory, higher PRF can increase total delivered ultrasound energy and angiogenic effect, but may also increase transducer heating. In some cases, a very low PRF may lead to insufficient cavitation and shear stress (and only a short-term vasodilation effect), while very high PRF may lead in some cases to transducer warming, lethal vascular damage (including possible dissection, stenosis, or thromboembolism), microhemorrhage, possible nerve damage, pain, fat or other tissue necrosis, apoptosis, and/or scar formation. In some embodiments, the PRF provided can be about, more than about, or no more than about 0.1 Hz, 0.5 Hz, 1 Hz, 1.5 Hz, 2 Hz, 2.5 Hz, 3 Hz, 3.5 Hz, 4 Hz, 4.5 Hz, 5 Hz, 6 Hz, 7 Hz, 8 Hz, 9 Hz, 10 Hz, 12 Hz, 14 Hz, 16 Hz, 18 Hz, 20 Hz, 25 Hz, 30 Hz, 35 Hz, 40 Hz, 45 Hz, 50 Hz, 60 Hz, 70 Hz, 80 Hz, 90 Hz, 100 Hz, 110 Hz, 120 Hz, or ranges incorporating any of the foregoing values. In some embodiments, systems and methods can provide a constant or variable PRF.

### Pulse Duration

In some embodiments, the pulse duration can be between about 1 μs (3 oscillations of 3 MHz) and about 100 ms, between about 1 ms and about 10 ms, between about 1 μs and about 100 μs, between about 100 μs and about 500 μs, between about 500 μs and about 1 ms, between about 1 ms and about 5 ms, between about 5 ms and about 20 ms, between about 10 ms and about 50 ms, between about 25 ms and about 100 ms in some embodiments, or overlapping ranges thereof. Not to be limited by theory, longer pulses can increase total delivered ultrasonic energy and likely angiogenic effect, but may also increase transducer heating. In some cases, a very low pulse duration may lead to insufficient cavitation and shear stress (and only a short-term vasodilation effect), while very high pulse durations may lead in some cases to transducer warming, lethal vascular damage (including possible dissection, stenosis, or thromboembolism), microhemorrhage, possible nerve damage, pain, fat or other tissue necrosis, apoptosis, and/or scar formation. In some embodiments, the pulse duration provided can be about, more than about, or no more than about 1 μs, 5 μs, 10 μs, 25 μs, 50 μs, 100 μs, 250 μs, 500 μs, 750 μs, 1 ms, 2 ms, 3 ms, 4 ms, 5 ms, 6 ms, 7 ms, 8 ms, 9 ms, 10 ms, 15 ms, 20 ms, 25 ms, 30 ms, 35 ms, 40 ms, 45 ms, 50 ms, 60 ms, 70 ms, 80 ms, 90 ms, 100 ms, 110 ms, 120 ms, or ranges incorporating any of the foregoing values. In some embodiments, systems and methods can provide a constant or variable pulse duration.

### Duty Factor

In some embodiments, the duty factor can be between about 0.1% and about 50%, such as between about 0.5% and about 2%, between about 0.1% and about 0.5%, between about 1% and about 5%, between about 2% and about 10%, between about 5% and about 20%, between about 20% and about 50%, or about 1% in some embodiments, or overlapping ranges thereof. Higher duty factor can increase total delivered ultrasonic energy and likely angiogenic effect, but may also increase transducer heating. In some cases, a very low duty factor may lead to insufficient cavitation and shear stress (and only a short-term vasodilation effect), while very high duty factors may lead in some cases to transducer warming, lethal vascular damage (including possible dissection, stenosis, or thromboembolism), microhemorrhage, possible nerve damage, pain, fat or other tissue necrosis, apoptosis, and/or scar formation. In some embodiments, the duty factor provided can be about, more than about, or no more than about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, or ranges incorporating any of the foregoing values.

For embodiments incorporating a phased array of transducers, as demonstrated in FIG. 10, each transducer could have a duty cycle that is up to about 1/(total number of transducers), for example in an array of 8 transducers, each may have up to about a 12.5% duty cycle. Each transducer could have equal duty cycles, or unequal duty cycles in some embodiments.

### Peak Negative Pressure

In some embodiments, the peak negative pressure (p⁻; greater p− can be associated with more shear stress and cavitation, and angiogenic effect, although high p− can theoretically lead to vascular damage) can be between about 2 MPa and about 20 MPa, between about 6 MPa and about 10 MPa, between about 2 MPa and about 4 MPa, between about 1.5 MPa and about 4 MPa, between about 1 MPa and about 4 MPa, between about 2.5 MPa and about 3.5 MPa, between about 3 MPa and about 5 MPa, between about 4 MPa and about 6 MPa, between about 5 MPa and about 7 MPa, between about 7 MPa and about 10 MPa or less than about 4 MPa in some embodiments. For clarity, the minus signs preceding the peak negative pressure disclosed herein are omitted—for example, a peak negative pressure of 4 MPa (can be denoted elsewhere as-4 MPa) as described herein is more negative than a peak negative pressure of 1 MPa (can be denoted elsewhere as-1 MPa). In some embodiments, the p⁻ may be selected to maximize sub-lethal cavitation. In some cases, a very low p⁻ may lead to insufficient cavitation and shear stress, while very high p⁻ may lead in some cases to transducer warming, lethal vascular damage (including possible dissection, stenosis, or thromboembolism), microhemorrhage, possible nerve damage, pain, fat or other tissue necrosis, apoptosis, and/or scar formation. In some embodiments, the p⁻ provided can be about, more than about, or no more than about 0.5 MPa, 1 MPa, 1.5 MPa, 2 MPa, 2.5 MPa, 3 MPa, 3.5 MPa, 4 MPa, 4.5 MPa, 5 MPa, 5.5 MPa, 6 MPa, 6.5 MPa, 7 MPa, 7.5 MPa, 8 MPa, 8.5 MPa, 9 MPa, 9.5 MPa, 10 MPa, 10.5 MPa, 11 MPa, 12 MPa, 13 MPa, 14 MPa, 15 MPa, 16 MPa, 17 MPa, 18 MPa, 19 MPa, 20 MPa, 21 MPa, 22 MPa, 25 MPa, or ranges incorporating any of the foregoing values.

### Acoustic Intensity

In some embodiments, the ultrasound parameters may be configured to provide an acoustic dose as calculated at the surface, or the target tissue as described herein to specifically promote angiogenesis and/or vasodilation. In some embodiments, the acoustic dose as calculated at either the surface, or by derated Iₛₚₜₐ of between about 250 mW/cm² and about 5,000 mW/cm², between about 250 mW/cm² and about 720 mW/cm², between about 720 mW/cm² and about 5000 mW/cm², between about 500 mW/cm² and about 1,000 mW/cm², between about 750 mW/cm² and about 1,500 mW/cm², between about 1 W/cm² and about 2 W/cm², between about 2 W/cm² and about 4 W/cm², between about 3 W/cm² and about 5 W/cm², or overlapping ranges thereof. Derating is a method of making acoustic measurements to account for attenuation in tissue.

In some embodiments, the ultrasound parameters can be configured to provide intensity at the surface, or a derated Isppa of between about 50 W/cm² and about 1000 W/cm², such as between about 50 W/cm² and about 190 W/cm², between about 190 W/cm² and about 1000 W/cm², between about 150 W/cm² and about 300 W/cm², between about 200 W/cm² and about 500 W/cm², or between about 500 W/cm² and about 1000 W/cm², or overlapping ranges thereof. In some embodiments, the intensity at the surface, or a derated Iₛₚₜₐ provided can be about, more than about, or no more than about 150 mW/cm², 200 mW/cm², 250 mW/cm², 300 mW/cm², 350 mW/cm², 400 mW/cm², 450 mW/cm², 500 mW/cm², 550 mW/cm², 600 mW/cm², 650 mW/cm², 700 mW/cm², 750 mW/cm², 800 mW/cm², 850 mW/cm², 900 mW/cm², 950 mW/cm², 1,000 mW/cm², 1,250 mW/cm², 1,500 mW/cm², 1,750 mW/cm², 2,000 mW/cm², 2,250 mW/cm², 2,500 mW/cm², 2,750 mW/cm², 3,000 mW/cm², 3,250 mW/cm², 3,500 mW/cm², 3,750 mW/cm², 4,000 mW/cm², 4,250 mW/cm², 4,500 mW/cm², 4,750 mW/cm², 5,000 mW/cm², or ranges incorporating any of the foregoing values. In some embodiments, the intensity can be, for example, between about 500 mW/cm² and about 5,000 mW/cm² or between about 1,000 mW/cm² and about 4,000 mW/cm².

In some embodiments, the ultrasound parameters can be configured to provide a mechanical index (MI, defined as MI=p⁻_{d}/√f, where p⁻_{d} is derated peak negative pressure and f is frequency) of between about 1 and about 10, such as no more than about 1.9, between about 2 and about 10, between about 1 and about 4, between about 4 and about 10, between about 1 and about 2, between about 2 and about 4, between about 3 and about 5, between about 4 and about 8, or between about 5 and about 10 in some embodiments, or overlapping ranges thereof. In some embodiments, the mechanical index provided can be about, at least about, or no more than about 0.5, 1, 2, 3, 4, 5, 6, 7, 8, 9, 10, 11, 12, or ranges incorporating any of the foregoing values.

In some embodiments, the system could be configured to deliver ultrasound energy in continuous wave (CW) mode, pulse wave (PW) mode, or both modes.

In some embodiments, the system can be configured to deliver energy with a surface intensity: vessel depth ratio to preferentially treat the target tissue (e.g., angiosome(s) in some embodiments). The surface intensity: vessel depth ratio can be, for example, about or less than about 0.75, 0.70, 0.65, 0.60, 0.55, 0.50, 0.45, 0.40, 0.35, 0.30, 0.25, 0.20, 0.15, or 0.10 W/cm³ in some embodiments, or ranges incorporating any two of the foregoing values, but in some cases at least about 0.05, 0.075, 0.10, 0.125, 0.15, 0.175, or 0.20 W/cm³.

In some embodiments, the surface intensity: vessel depth ratio is between about 0.10 W/cm³ and about 0.60 W/cm³, between about 0.10 W/cm³ and about 0.55 W/cm³, between about 0.125 W/cm³ and about 0.50 W/cm³, or between about 0.20 W/cm³ and about 0.50 W/cm³. Not to be limited by theory, such ratios among others have unexpectedly been found to advantageously treat PAD and other indications as described herein in some cases by focused ultrasound delivery to the target tissue while minimizing off-target effects.

In some embodiments, the intensity to surface area of the skin overlying the target tissue (e.g., angiosome(s)) can be about, less than about, or at least about 5:1, 4.5:1, 4:1, 3.5:1, 3:1, 2.5:1, 2:1, 1.5:1, 1:1 or less or ranges incorporating any two of the aforementioned values. Such ratios have unexpectedly been found to advantageously treat PAD and other indications as described herein in some cases.

In some embodiments, the maximum power delivered can be, in some cases, between about 30 Amps and about 70 Amps, between about 40 Amps and about 60 Amps to foot angiosomes, or about or no more than about 75, 70, 65, 60, 55, 50, 45, 40, 35, 30, 25 Amps, or ranges incorporating any two of the aforementioned values. In some embodiments, for calf angiosomes, the maximum power delivered can be, for example, between about 100 Amps and about 250 Amps, between about 125 Amps and about 225 Amps, or about or less than about 250, 245, 240, 235, 230, 225, 220, 215, 210, 205, 200, 195, 190, 185, 180, 175, 170, 165, 160, 155, 150, 145, 140, 135, 130, 125, 120, 115, 110, 105, 100 Amps, or less, or ranges incorporating any two of the aforementioned values.

In some embodiments, the surface power/intensity ratio of the ultrasonic energy delivered can be about, at least about, or no more than about 1, 2, 3, 4, 5, 6, 7, 8, 9, 10, 11, 12, 13, 14, 15, 16, 17, 18, 19, 20, 21, 22, 23, 24, or 25 cm², or ranges incorporating any two of the aforementioned values and selected to better focus ultrasound to the target tissue. In some embodiments, the surface power/intensity ratio can be, for example, between about 3 cm² and about 25 cm², between about 3 cm² and about 5 cm², between about 15 cm² and about 25 cm², or less than about 25, 20, 15, 10, 5 cm², or less.

In some embodiments, the therapeutic energy can be focused to a particular depth depending on the desired target tissue, e.g., angiosome(s). An angiosome is a 3-dimensional anatomic unit of tissue (including skin, subcutaneous tissue, fascia, muscle, and bone) fed by a source artery and drained by specific veins. The entire body can be divided into 40 angiosomes. The lower leg, below the knee and including the foot includes six angiosomes. The posterior tibial artery feeds three angiosomes (the medial calcaneal artery angiosome; the medial plantar artery angiosome; and the lateral plantar artery angiosome), the anterior tibial feeds one (the dorsalis pedis artery angiosome), and the peroneal artery feeds two (the lateral calcaneal artery angiosome and the anterior perforating branch artery angiosome). Any number of the aforementioned angiosomes can be treated to create a therapeutic effect (e.g., increased blood flow, such as via angiogenesis and/or vasodilation) using systems and methods as disclosed herein. The posterior tibial artery gives rise to a calcaneal branch, which supplies the medial ankle and lateral plantar heel, a medial branch that feeds the medial plantar instep/arch, and a lateral branch that supplies the lateral forefoot, plantar midfoot, and entire plantar forefoot. The anterior tibial artery continues on to the dorsum of the foot as the dorsalis pedis artery. The peroneal artery supplies the lateral ankle and plantar heel via the calcaneal branch and the anterior upper ankle via an anterior branch. As such, directing therapeutic energy to 1, 2, 3, 4, 5, 6, or more angiosomes, such as in the lower extremity below the knee and/or foot for example can advantageously promote angiogenesis, vasodilation and other benefits as described for example herein. Non-limiting examples of angiosomes to be targeted can be found in the Figures, for example, FIG. 3A, which schematically illustrates six angiosomes.

For calf angiosomes, for example, in some embodiments the energy can be focused to a vessel depth of, for example, between about 3 cm and about 9 cm, such as between about 4 cm and about 8 cm, or between about 4.5 cm and about 7 cm. In some embodiments, dorsal or plantar foot angiosomes, for example, the energy can be focused to a vessel depth of, for example, between about 1 cm and about 4 cm, such as between about 1.5 cm and about 3.5 cm, or between about 2 cm and about 3 cm. In some embodiments, the energy can be focused to a vessel depth of about, at least about, or no more than about 1 cm, 1.5 cm, 2 cm, 2.5 cm, 3 cm, 3.5 cm, 4 cm, 4.5 cm, 5 cm, 5.5 cm, 6 cm, 6.5 cm, 7 cm, 7.5 cm, 8 cm, 8.5 cm, 9 cm, 9.5 cm, 10 cm, 11 cm, 12 cm, 15 cm, or ranges incorporating any two of the aforementioned values.

As some non-limiting examples, in some embodiments delivering TUS ultrasonic energy to a calf or foot angiosome(s) at a frequency of between about 1 MHz and about 3 MHz, a peak negative pressure of between about 2 MPa and about 4 MPa, energy delivery of between about 1 W/cm² and about 4 W/cm² at the target tissue level, and a surface intensity: vessel depth ratio between about 0.10 W/cm³ and about 0.60 W/cm³, for a cumulative total of about or at least about 10, 20, 30, 40, 50, 60, or more cumulative minutes per week for at least about 1, 2, 3, 4, 5, 6, 7, 8, 9, 10, or more weeks can surprisingly and unexpectedly promote angiogenesis, vasodilation, and/or collaterogenesis in some cases.

All of the processes described herein may be embodied in, and fully automated via, software code modules, including one or more specific computer-executable instructions, that are executed by a computing system. The computing system may include one or more computers or processors. The code modules may be stored in any type of non-transitory computer-readable medium or other computer storage device. Some or all the methods may be embodied in specialized computer hardware.

Many other variations than those described herein will be apparent from this disclosure. For example, depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. A computing environment can include any type of computer system, including, but not limited to, a computer system based on a microprocessor, a mainframe computer, a digital signal processor, a portable computing device, a device controller, or a computational engine within an appliance, to name a few.

Conditional language such as, among others, “can,” “could,” “might,” or “may,” unless specifically stated otherwise, are otherwise understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within 10% of, within 5% of, within 1% of, within 0.1% of, and within 0.01% of the stated amount.

Any process descriptions, elements or blocks in the flow diagrams described herein and/or depicted in the attached FIGS should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B, and C” can include a first processor configured to carry out recitation A working in conjunction with a second processor configured to carry out recitations B and C.

1.-26. (canceled)

27. A method, comprising:
providing a patient interface configured to be worn on a limb of a patient, the patient interface comprising a transducer array and a memory;
determining, for each transducer of the transducer array, an efficiency value, wherein each efficiency value indicates an output power of a transducer of the transducer array at a particular input drive voltage; and
storing the efficiency value of each transducer of the transducer array in the memory of the patient interface, wherein the stored efficiency values are configured to be retrieved by a therapeutic ultrasound device to determine a transducer activation sequence of the transducers from the highest efficiency value to the lowest efficiency value, and to provide ultrasonic energy from each transducer in the transducer array in order of the activation sequence.

28. The method of claim 27, further comprising providing the efficiency value for each transducer of the transducer array to a therapeutic ultrasound system.

29. The method of claim 27, wherein determining the efficiency value comprises determining a ratio of the output power to the particular input drive voltage.

30. A patient interface, comprising:
a connector, configured to connect the patient interface to a therapeutic ultrasound system, wherein the patient interface is configured to be worn on a limb of a patient;
a transducer array, comprising a plurality of transducers, wherein the transducer array is in electrical communication with the connector; and
a memory, configured to store information associated with each transducer of the transducer array, and wherein the memory is in electrical communication with the connector; and
wherein the information associated with each transducer of the transducer array comprises a plurality of efficiency values, each efficiency value indicating an output power of a transducer of the transducer array at a particular input drive voltage, wherein the information stored in the memory is configured to be retrieved by a therapeutic ultrasound device to determine a transducer activation sequence of the transducers from the highest efficiency value to the lowest efficiency value, and to provide ultrasonic energy from each transducer in the transducer array in order of the activation sequence.

31. The patient interface of claim 30, wherein the efficiency value comprises a ratio of the output power to the particular input drive voltage.

32. A method, comprising:
receiving from a memory, an efficiency value associated with each transducer of a transducer array, wherein the efficiency value indicates an output power of a transducer of the transducer array at a particular input drive voltage;
determining a transducer activation sequence based upon the efficiency values of each transducer of the transducer array, wherein the activation sequence indicates an order of the transducers of the transducer array from a highest efficiency value to a lowest efficiency value, and
sequentially activating each transducer of the transducer array according to the transducer activation sequence such that a most efficient transducer of the transducer array is activated first and a least efficient transducer of the transducer array is activated last.

33. (canceled)

34. The method of claim 32, wherein activating comprises providing a transducer with a control signal having a specified frequency for a specified duration.

35. The method of claim 32, wherein activating each transducer occurs during a ramp-up period.

36. (canceled)

37. (canceled)

38. The method of claim 32, further comprising storing the transducer activation sequence in a memory.

39. The method of claim 32, wherein sequentially activating each transducer comprises sequentially driving each transducer of the transducer array at an initial applied drive voltage that is less than a final applied voltage.

40. The method of claim 39, further comprising determining an increased applied voltage over a ramp-up time period, wherein the increased applied voltage is greater than the initial drive voltage and driving the transducer of the transducer array at the increased applied voltage during the ramp-up time period.

41. The method of claim 40, wherein the ramp-up time period is 3 min, 5 min, 7 min, 10 min, 15 min or 30 min.

42. The method of claim 40, wherein determining an increased applied voltage comprises increasing the initial applied voltage by 5%, 10%, 15%, 20%, 25%, 30%, 35%, 40%, 45%, or 50% of the initial applied voltage.

43. The method of claim 40, wherein increasing the initial applied voltage comprises increasing the initial applied voltage by 5%, 10%, 15%, 20%, 25%, 30%, 35%, 40%, 45%, or 50% of the final applied voltage.

44. The method of claim 40, wherein increasing the initial applied voltage comprises increasing the initial applied voltage in constant amounts in steps during the ramp-up time period.

45. The method of claim 40, wherein increasing the initial applied voltage comprises increasing the initial applied voltage in non-constant amounts in steps during the ramp-up time period.

46.-63. (canceled)
